(19) United States
(12) Patent Application Publication
(10) Pub. No.: US 2009/0018108 A1
(43) Pub. Date: Jan. 15, 2009

(54) Treating skin hyperpigmentation with dermatological compositions comprising hydroquinone, fluocinolone acetonide and tretinoin

(75) Inventors: Isabelle Pelisson, Vallauris (FR); Andre Jomard, Saint Vallier De Thiey (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., Cham (CH)

(21) Appl. No.: 12/155,938

(22) Filed: Jun. 11, 2008

Related U.S. Application Data
(63) Continuation of application No. PCT/FR2006/051295, filed on Dec. 6, 2006.

(30) Foreign Application Priority Data
Dec. 14, 2005 (FR) 0512680

(51) Int. Cl. A61K 31/58; A61P 17/06; A61P 17/00

(52) U.S. Cl. 514/171

(57) ABSTRACT

Hyperpigmentary skin disorders, notably those associated with psoriasis or eczema, are prevented/treated by administering to individuals in need of such treatment, thus effective amounts of medicaments containing a combination of fluocinolone acetonide, hydroquinone and tretinoin, formulated into a physiologically acceptable medium therefor.

### CROSS-REFERENCE TO PRIORITY/PCT APPLICATIONS

This application claims priority under 35 U.S.C. § 119 of FR 0512680, filed Dec. 14, 2005, and is a continuation of PCT/FR 2006/051295, filed Dec. 6, 2006 and designating the United States (published in the French language on Jun. 21, 2007 as WO 2007/068844 A1; the title and abstract were also published in English), each hereby expressly incorporated by reference in its entirety and each assigned to the assignee hereof.

### BACKGROUND OF THE INVENTION

1. Technical Field of the Invention

The present invention relates to dermatological compositions comprising a combination of hydroquinone, fluocinolone acetonide and tretinoin, for treating skin hyperpigmentation following, notably, psoriasis or eczema.

2. Description of Background and/or Related and/or Prior Art

Psoriasis is a benign, chronic, erythematosquamous dermatosis that affects certain regions with predilection (elbow, knees, sacral region and scalp), and sometimes the entire body, consisting of clearly limited plaques or sheets, covered with thick, white, nacreous squames, which are removed by scratching, revealing a shiny red and bleeding surface.

This condition can become generalized and assume the very specific appearance of pustular psoriasis.

Anatomopathological examination shows hyperkeratosis with parakeratosis and acanthosis of the epidermis linked to an excessive proliferation of keratinocytes. In addition, the epidermis is the seat of microabcesses containing polymorphonuclear cells.

In particular, palmoplantar psoriasis can produce keratoderma in islands or diffuse keratoderma. Moreover, palmoplantar psoriasis is one of the forms of palmoplantar keratoderma of various causes, and it is difficult to distinguish it from chronic eczema.

Psoriasis can therefore be more or less serious. The serious forms of psoriasis are called erythrodermic psoriasis, arthropathic psoriasis and pustular psoriasis.

In addition, psoriasis can promote the appearance of post-inflammatory hyperpigmentation.

The treatments for psoriasis depend not only on the seriousness and on the extent of the lesions, but also on the damage in functional, aesthetic, occupational and relationship terms, on the psychological effect of the disease and on the patient's desire for remission.

The current treatments do not result in the condition being definitively cured, but more or less complete, transient disappearance of the lesions is obtained.

In particular, dermocorticosteroids are commonly used in the form of ointments. Creams are reserved for the folds and lotions for the scalp.

Among these dermocorticosteroids, exemplary is fluocinolone acetonide, for example marketed under the trademark Fluoderm®.

Retinoids such as acitretin may also be used, in particular in the treatment of pustular psoriasis.

Moreover, it is also known that the combination of a desquamating agent such as alpha-hydroxy acids and of a corticosteroid such as betamethasone reveals a synergistic action in the treatment of psoriasis, in particular of psoriasis of the scalp.

Eczema is a very common skin condition characterized by pruriginous erythematovesicular lesions that are often poorly limited, corresponding histologically to foci of epidermal spongiosis, and the allergic mechanism of which involves delayed cellular immunity and humoral immunity, in a complex manner.

Eczema may be acute, chronic or constitutional.

Acute eczema progresses in four successive phases:
the erythematous phase,
the vesicular phase,
the weeping phase, and
the desquamative phase followed by recovery with scars.

The lesions are highly pruriginous.

Chronic eczema can have a dry form (the skin exhibits poorly delimited red plaques covered with scabs, with variable desquamation), a lichenified form (plaques of thick skin with fissures running through them), or a dysidrotic form: vesicles on the lateral faces of the fingers, which, when they break, can form scabs or fissures.

Constitutional eczema is a skin disease which progresses in a chronic manner or by recurring attacks.

Eczema can promote the appearance of post-inflammatory hyperpigmentation.

The treatment often uses local cortico therapy, i.e., dermocorticosteroids.

### SUMMARY OF THE INVENTION

It has now surprisingly been discovered that the combination of hydroquinone, fluocinolone acetonide and tretinoin is useful for treating skin hyperpigmentations caused by psoriasis or by eczema.

The present invention therefore features the administration of a combination of fluocinolone acetonide, hydroquinone and tretinoin, formulated as a medicament, for preventing and/or treating hyperpigmentary skin disorders associated with psoriasis or with eczema.

The term “hyperpigmentary disorders associated with psoriasis” means any skin hyperpigmentation resulting from this pathology, in particular post-inflammatory hyperpigmentations or pigmentary acanthoses.

### DETAILED DESCRIPTION OF BEST MODE AND SPECIFIC/PREFERRED EMBODIMENTS OF THE INVENTION

Advantageously, the subject medicaments are useful for preventing and/or treating hyperpigmentary disorders associated with psoriasis, whether regime or regimen; preferably, such medicaments are useful for preventing and/or treating psoriasis of the scalp.

Advantageously, the medicaments according to the present invention are formulated for topical application.

The medicaments according to the invention also comprise a physiological acceptable medium, i.e., a medium which is compatible with the skin, including the scalp, the mucous membranes, the hair, body hair and/or the eyes, and can constitute a dermatological composition.

This composition may also comprise any of the constituents normally present in the type of application envisioned.

The medicaments according to the present invention may comprise a large variety of additional components; they may in particular be absorbents, abrasives, anti-acne agents, anti-foams, anti-microbial agents, antioxidants, binders, biological additives, buffers, chelating agents, dyes, cosmetic astringents, cosmetic biocides, external analgesics, film-forming agents, fragranced components, opacifiers, plasticizers, preservatives, other depigmenting agents, emollients, skin-protecting agents, solvents, solubilizing agents, surfactants, ultraviolet light-absorbing agents, sunscreens, viscosity-increasing agents (aqueous or non-aqueous), humectants, sequestering agents, etc.

Those skilled in the art will obviously take care to select the possible additional compounds and/or the amount thereof in such a manner that the advantageous properties of the medicaments according to the present invention are not completely or not substantially reduced by the addition envisioned.

Hydroquinone is a known depigmenting agent. It is prepared by reduction of p-benzoquinone with sodium bisulfite. The chemical name for hydroquinone is 1,4-benzenediol.

Advantageously, the hydroquinone is present in the medicament at a concentration of from 1% to 10% by weight, advantageously from 2% to 7%, and more advantageously at a concentration of 4% by weight, relative to the total weight of the medicament.

Tretinoin is an all-trans retinoic acid formed by oxidation of the aldehyde group of retinene, to a carboxyl group. The chemical name for tretinoin is: (all-E)-3,7-dimethyl-9-(2,6,6-trimethyl-1-cyclohexen-1-yl)-2,4,6,8-non-atetraenoic acid. It is highly reactive to light and to moisture and is known as a keratolytic agent.

In a specific embodiment, the tretinoin is present in the medicaments according to the present invention at a concentration of from 0.025% to 2% by weight, advantageously from 0.025% to 1% by weight, even more advantageously of approximately 0.05% by weight, relative to the total weight of the medicament.

Fluocinolone acetonide is a synthetic fluorinated corticosteroid for topical dermatological use, and it is useful as an anti-inflammatory. Its chemical name is (6,11,16)-6,9-difluoro-11,21-dihydroxy-16,17-[(1-methylethylidene)bis(oxy)]pregna-1,4-diene-3,20-dione. It is a white crystalline powder which has no odor and is light-stable.

In a specific embodiment, the fluocinolone acetonide is present in the medicaments according to the present invention at a concentration of from 0.005% to 0.1% by weight, advantageously from 0.005% to 0.05% by weight, even more advantageously of approximately 0.01% by weight, relative to the total weight of the medicament.

Advantageously, the medicaments according to the present invention contain sodium metabisulfite in order to prevent oxidation of the hydroquinone.

Additional components may be present in the medicaments according to the present invention in an amount of from 0.001% to 20% by weight, relative to the total weight of the medicament.

The medicaments according to the present invention may be provided in any of the galenical forms normally used in the dermatology field. Preferably, the composition according to the present invention is in the form of a cream.

The creams may advantageously be prepared as indicated in WO 2004/037201, by means of a method comprising the steps of:
a) mixing the hydrophilic compounds with water to form an aqueous phase;
b) mixing the hydrophobic compounds to form a hydrophobic phase;
c) mixing the hydrophobic and hydrophilic phases to form a two-phase mixture, and
d) adding an emulsifier to the two-phase phase to form an emulsion.

In addition, they may contain other customary ingredients of creams and may be formulated in a manner well known to those skilled in the art.

Advantageously, the medicaments according to the present invention contain at least one inactive ingredient selected from among butylated hydroxytoluene, cetyl alcohol, citric acid, glycerol, glyceryl stearate, magnesium aluminum silicate, methyl gluceth-10, methylparaben, PEG-100 stearate, propylparaben, purified water, sodium metabisulfite, stearic acid and stearyl alcohol.

Advantageously, the medicaments according to the present invention are the cream Tri-Luma® marketed by Galderma, as presented in Example 1.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Composition of the Cream Tri-Luma®

The cream contains, as percentage by weight relative to the total weight:

| | |
|---|---|
| magnesium aluminum silicate | 3.00% |
| butylated hydroxytoluene | 0.04% |
| cetyl alcohol | 4.00% |
| stearic acid | 3.00% |
| stearyl alcohol | 4.00% |
| methylparaben | 0.18% |
| propylparaben | 0.02% |
| Arlacel ® 165 [glyceryl stearate and | 3.50% |
| glyceryl monostearate and PEG-100 stearate] | |
| methyl gluceth-10 | 5.00% |
| glycerol | 4.00% |
| tretinoin | 0.05% |
| fluocinolone acetonide | 0.01% |
| citric acid | 0.05% |
| hydroquinone | 4.00% |
| sodium metabisulfite | 0.20% |
| purified water | 68.95% |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for preventing and/or treating a hyperpigmentary skin disorder, comprising administering to an individual in need of such treatment, a thus effective amount of a medicament which comprises a combination of fluocinolone acetonide, hydroquinone and tretinoin, formulated into a physiologically acceptable medium therefor.

2. A regime or regimen for preventing and/or treating a hyperpigmentary skin disorder, comprising topically applying onto the skin of an individual in need of such treatment, a thus effective amount of a medicament which comprises a combination of fluocinolone acetonide, hydroquinone and tretinoin, formulated into a topically applicable, physiologically acceptable medium therefor.

3. The regime or regimen as defined by claim 1, wherein the hydroquinone is present at a concentration of from 1% to 10% by weight, relative to the total weight of the medicament.

4. The regime or regimen as defined by claim 3, wherein the hydroquinone is present at a concentration of 4% by weight, relative to the total weight of the medicament.

5. The regime or regimen as defined by claim 3, wherein the tretinoin is present at a concentration from 0.025% to 2% by weight, relative to the total weight of the medicament.

6. The regime or regimen as defined by claim 5, wherein the tretinoin is present at a concentration of approximately 0.05% by weight, relative to the total weight of the medicament.

7. The regime or regimen as defined by claim 5, wherein the fluocinolone acetonide is present at a concentration from 0.005% to 0.1% by weight, relative to the total weight of the medicament.

8. The regime or regimen defined by claim 7, wherein the fluocinolone acetonide is present at a concentration of approximately 0.01% by weight, relative to the total weight of the medicament.

9. The regime or regimen as defined by claim 1, said medicament being formulated as a cream.

10. The regime or regimen as defined by claim 1, said medicament comprising the following composition, in percentages by weight relative to the total weight thereof:

| | |
|---|---|
| magnesium aluminum silicate | 3.00% |
| butylated hydroxytoluene | 0.04% |
| cetyl alcohol | 4.00% |
| stearic acid | 3.00% |
| stearyl alcohol | 4.00% |
| methylparaben | 0.18% |
| propylparaben | 0.02% |
| Arlacel ® 165 [glyceryl stearate and | 3.50% |
| glyceryl monostearate and PEG-100 stearate] | |
| methyl gluceth-10 | 5.00% |
| glycerol | 4.00% |
| tretinoin | 0.05% |
| fluocinolone acetonide | 0.01% |
| citric acid | 0.05% |
| hydroquinone | 4.00% |
| sodium metabisulfite | 0.20% |
| purified water | 68.95% |

11. The regime or regimen as defined by claim 1, said hyperpigmentary skin disorder being associated with psoriasis.

12. The regime or regimen as defined by claim 1, said hyperpigmentary skin disorder being associated with eczema.

13. The regime or regimen as defined by claim 1, said medicament comprising a hydroquinone antioxidant.

14. The regime or regimen as defined by claim 1, said medicament comprising at least one inactive constituent selected from the group consisting of butylated hydroxytoluene, cetyl alcohol, citric acid, glycerol, glyceryl stearate, magnesium aluminum silicate, methyl gluceth-10, methylparaben, PEG-100 stearate, propylparaben, purified water, sodium metabisulfite, stearic acid and stearyl alcohol.
